(19) United States
(12) Patent Application Publication
(10) Pub. No.: US 2021/0180051 A1
(43) Pub. Date: Jun. 17, 2021

(54) METHODS AND SYSTEMS TO AMPLIFY SHORT RNA TARGETS

(71) Applicant: Paragon Genomics, Inc., Hayward, CA (US)

(72) Inventors: Zhitong LIU, Foster City, CA (US); Chenyu LI, Hayward, CA (US); David DEBRUYNE, Hayward, CA (US); Julia SPENCER, Hayward, CA (US)

(21) Appl. No.: 16/716,487

(22) Filed: Dec. 16, 2019

(51) Int. Cl. C12N 15/10; C12Q 1/6874

(52) U.S. Cl. CPC C12N 15/1096; C12Q 1/6874; C12N 15/101; C12N 15/1013

(57) ABSTRACT

Methods, compositions and systems to amplify targets from RNA samples. The methods comprise designing target-specific primers, converting RNA into cDNA by reverse transcription, adding a universal primer by using template switching, and amplifying the targets by using multiplex PCR. The methods, compositions and systems described herein may further include, or include the use of, next generation sequencing (NGS) to analyze the sequences and various mutations of the amplified targets.

### CROSS REFERENCE TO RELATED APPLICATIONS

This patent application may be related to U.S. patent application Ser. No. 15/290,981, filed on Oct. 11, 2016, which claims priority as a continuation-in-part to U.S. patent application Ser. No. 15/041,644, filed on Feb. 11, 2016, now U.S. Pat. No. 9,464,318, and titled “METHODS AND COMPOSITIONS FOR REDUCING NON-SPECIFIC AMPLIFICATION PRODUCTS”, which claims priority to U.S. provisional patent applications: U.S. Provisional Patent Application No. 62/114,788, titled “A METHOD FOR ELIMINATING NONSPECIFIC AMPLIFICATION PRODUCTS IN MULTIPLEX PCR” and filed on Feb. 11, 2015; and U.S. Provisional Patent Application No. 62/150,600, titled “METHODS AND COMPOSITIONS FOR REDUCING NON-SPECIFIC AMPLIFICATION PRODUCTS” and filed Apr. 21, 2015. Each of these applications is herein incorporated by reference in its entirety.

This patent application may also be related to international patent application no. PCT/US2018/013143 and U.S. patent application Ser. No. 15/867,031, filed on Jan. 10, 2018, which claims priority to U.S. provisional patent applications: U.S. Provisional Patent Application No. 62/444,704, titled “METHODS AND COMPOSITIONS FOR REDUCING REDUNDANT MOLECULAR BARCODES CREATED IN PRIMER EXTENSION REACTIONS” and filed on Jan. 10, 2017. Each of these applications is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods, compositions, systems and kits described herein relate to the amplification of nucleotide sequences. In particular, the methods, compositions and systems described herein relate to increasing the efficiency of amplifying multiple short RNA fragments. The methods, compositions and systems described herein may include analyzing the resulting DNA library by high throughput sequencing (next generation sequencing, NGS).

### BACKGROUND

The advances in next-generation sequencing (NGS) technology have provided a new approach to detect genomic structural alterations involving chromosomal breakage and rearrangement. These structural alterations often result in gene fusion mutations. Fusion mutations happen frequently in a number of cancers, for example, EML4-ALK fusion in lung cancer, BCR-ABL1 fusion in chronic myeloid leukemia, EWSR1-FLI1 fusion in Ewing's sarcoma, etc. There is a huge demand in detecting chromosomal alterations in RNA level, where these alterations are spliced into mRNA. Specifically, the demand is to detect fusion mutations in FFPE samples, where RNA is broken into short fragments; or in fine-needle aspiration RNA samples, which contains very low amount of RNA.

It is challenging to amplify short RNA fragments. Firstly, the poly(A) tail of mRNA is lost in the majority of RNA fragments. Oligo(dT) primer therefore cannot be used in reverse transcription to convert RNA into cDNA, unless and poly(A) tail is added onto each RNA fragment. Random hexamers thus become the natural choice used to synthesizing cDNA. Secondly, adding a poly(A) tail onto each RNA fragment requires several steps, involving 3′ end modification, poly(A) tailing and purification. The efficiency of each step and the inevitable loss of RNA in purification discourages the use of poly(A)-tailing method. Thus, the random hexamer method becomes the most promising one. However, the consequence of using random hexamers is the production of even shorter cDNA fragments. It is challenging to amplify and detect mutations existing on a short cDNA fragments by PCR, since a pair of target-specific primers has to anneal simultaneously to the short cDNA fragment. Therefore, some cDNA fragments that harbor only one of the primers may not be amplified by regular PCR method, leading to loss of positive signal, or skewed positive frequency of the detected mutations.

Removing ribosomal RNA is another challenge in amplifying minute amounts of RNA fragments. Ribosomal RNA occupies more than 80% in total RNA. Ribosomal RNA fragments are entirely converted into cDNA fragment when random hexamers are used in reverse transcription. In the downstream target amplifications by PCR, the target specific primers may nonspecifically anneal to cDNA derived from ribosomal RNA, given its high concentrations in the reaction. In the worst case, Ribosomal RNA contamination may occupy from 20% to 90% in the final product. It is one of the serious problems in amplifying cDNA converted by random hexamers from short RNA fragments.

Technologies with one target-specific primer appear more suitable for amplify short RNA fragments. One primer approaches usually use a variety of methods to add a universal primer at one or both ends of the cDNA fragments. The cDNA fragment can then be amplified with the universal primer and a target specific primer. Historically, it has been challenging to develop one-primer technologies. Previously, a universal adapter is ligated onto both ends of the cDNA fragment. Given the low efficiency of ligation reactions and the loss of DNA in the transitions of multiple steps, the quality final library is usually low, resulting in the biased representation of mutation frequencies and the requirement of huge amounts of RNA samples. Other technologies, such as 5′-race, 3′-race, template switching, also suffer low efficiency of adding a universal primer onto the 5′ or 3′ end of cDNA fragments.

### SUMMARY OF THE DISCLOSURE

Described herein are single-primer technology that uses template switching to add a universal primer at 3′ end of newly synthesized cDNA from short RNA fragments. Upon removing the remaining primers and non-specific products in reverse transcription, the efficiency of template switching and reverse transcription may be improved as described herein, leading to high production of cDNA containing the universal primer. These methods and apparatuses (e.g., kits, systems, etc.) may also remove the ribosomal RNA in the final product. The cDNA may be used in multiplex PCR to amplify a plurality of targets. The methods and systems described herein allow the making of clean libraries from short RNA fragments. It may generate high quality libraries of over 90% on-target rate and less than 1% ribosomal RNA rate, and may be used to detect known fusion mutations by NGS sequencing.

In general, described herein are methods, compositions and systems for amplifying short RNA fragments. These methods, compositions and systems may also include reverse transcription and template switching, and a template-dependent multiplex primer extension reaction. These methods, compositions and systems may be useful in any reactions in which a plurality of primers may be used. For example, the methods, compositions and systems described herein may be particularly well suited for use with high throughput sequencing (next generation sequencing, NGS).

Described herein are methods, compositions and systems (e.g., kits, etc.) that use resolvase or a variety of single-stranded DNA specific exonucleases to remove primers and non-specific products after reverse transcription and multiplex PCR. The nuclease treatment, also described as “digestion” or “digestion step” in the methods and systems described herein improves the efficiency of reverse transcription and template switching and reduces the ribosomal RNA content to below 1%. It also makes the multiplex PCR possible by reducing the non-specific products to negligible levels.

For example, described herein are methods, compositions and systems for amplifying short RNA fragments in a template-dependent multiplex primer extension reaction. These methods and apparatuses (e.g., systems, kits, etc.) for performing them may include one or more enzymes to synthesize cDNA, and the amplification of the cDNA. The simplicity of the method enables high efficiency and detection of rare mutations from low amount of RNA samples.

It requires fewer steps to use random hexamers to convert short RNA fragment into cDNA than using oligo(dT) primer after adding a poly(A)-tail onto each RNA molecules. This Random hexamer method thus has higher efficiency theoretically. However, the random hexamer method results in even shorter cDNA fragments. This may make it difficult to amplify targets from the cDNA fragments by PCR, because one or both primers may not exist on the cDNA fragments. It thus becomes clear that there is a great advantage if the cDNA fragments can be amplified with one target specific primer. The methods and systems described herein use template switching to add a universal primer onto the 3′ end of the newly synthesized cDNA, thus enabling the short cDNA fragments being amplified with a universal primer and a target specific primer in the PCR reaction.

Template switching methods traditionally have low efficiency in adding 3′ universal primers, manifested by the fact that the resulting cDNA has been a poor template for downstream PCR. The yield of cDNA with the universal primer was low, and some targets were missing and under-represented. The methods and systems described herein teach that the quality of the downstream PCR is improved significantly when the reverse transcription-template switching reaction is treated with resolvase or single-stranded DNA specific nucleases. After the treatment, the remaining primers and non-specific product in reverse transcription-template switching reaction are removed. The resulting cDNA is successively used in multiplex PCR reactions. By using CleanPlex® multiplex PCR technology, the methods and systems described herein may be used to amplify a plurality of targets uniformly. A plurality of target-specific primers (e.g., >6, >10, >100, >1000, >10,000, etc.) can be used. The amplification products can be further amplified while sample barcodes and sequencing adapters are added. The final library can then be used in downstream analysis, such as NGS sequencing.

In the methods described above, the amplification of the target is not limited by the length of the cDNA fragments, or the requirement of the presence of two primer sites on a short cDNA fragment is eliminated. Any targets, short and long, that harbor one primer site, are amplified. These methods and systems described herein thus allows for amplifying and detecting target signals with significantly higher sensitivity from a limited amount of starting material. Further, these methods and systems allow for amplifying and detecting structural change of DNA, such as fusion genes. By modeling the efficiencies of amplifying short DNA fragments, we found the efficiencies of the single-primer amplification method are significantly higher than those of PCR method (FIG. 2). For examples, amplifying a 20 bp target on DNA fragments of 160 bp in length, single-primer method has an efficiency of 72%, while PCR has 56%.

Thus, the methods and apparatuses, including compositions and kits, described herein can amplify a plurality of target short RNA fragments. The length of the shortest target RNA fragment can be 30 bases on average. The length of the target RNA fragments can be 30-200, 40-200, 50-200, 60-200, 70-200, 80-200, 90-200, 100-200 bases, etc., in length. Or the length of the target RNA fragments can be 30-1000, 40-1000, 50-1000, 60-1000, 70-1000, 80-1000, 90-1000, 100-1000 bases, etc., in length. Or the length of the target RNA fragments can be any numbers of combinations as long as the shortest one is equal or longer than the shortest primer, and the longest one is equal or shorter than the length that can be transcribed by RNA polymerase and reverse transcribed by reverse transcriptase.

In general, the target nucleic acids may comprise total RNA, mRNA, or FFPE RNA, for example, RNA purified from Formalin-fixed, Paraffin-embedded (FFPE) tissue samples (FFPE RNA), cell-free RNA (cfRNA) or synthetic RNA.

RNA fragments are denatured in DEPC-treated water in the presence of random hexamers and dNTP, followed by chilling on ice to anneal the random hexamers into RNA. Then a template switching primer, a reverse transcriptase and buffer are added into the reaction. The reverse transcriptase should support template switching. After incubation at appropriate temperature for sufficient time, cDNA is synthesized and a universal adapter is added at the 3′ end by template switching. The remaining primers and non-specific products are degraded by treating the reaction with CleanPlex® Digestion Reagent (CleanPlex® Multiplex PCR Kit, Paragon Genomics). The reaction was then stopped and the resulting cDNA was purified.

Reverse transcription and template switching are carried out simultaneously in the same reaction. Any reverse transcriptase that has template switching activity can be used. These reverse transcriptases include, but not limited to, Superscript II, Protocsript II, SMARTScribe, Maxima H-, RevertAid, EnzScript, GoScript, RevertUP II, MMLV Point Mutant. The 5′ end portion of the template switching primer is a universal primer, which is used in downstream target specific amplification. The 3′ end is rGrGrG that allows reverse transcriptase to continue synthesizing the universal primer portion of cDNA. Template switching primer may be used at concentrations from 2 to 10 μM. 20 to 200 units of reverse transcriptase may be used in 20 μl reactions. The reverse transcription may be carried out by first incubation at a lower temperature for 10 to 20 minutes, followed by incubation at a higher temperature for 60 to 90 minutes. The lower temperature may be 8 to 25° C., the higher temperature may be 42 to 55° C.

CleanPlex® Digestion Reagent is added directly into the above reaction to remove primer and non-specific products. Finally, the cDNA is purified and ready for downstream target specific amplification.

As mentioned, a template-dependent primer extension reaction may be further included to enrich a plurality of specific targets from the above cDNA fragments. The template-dependent primer extension reaction may include any method involving a plurality of oligonucleotides as primers. The length of primers may be from 16-100 nucleotides; the length of amplicons may be from 16 bp-100,000 bp.

The target-specific primers may comprise any appropriate plurality of primers or pairs of primers, such as 7 primers or pairs of primers or more (e.g., at least 7 primers or pairs of primers) of target-specific primers, such as 10 primers or pairs of primers or more (e.g., at least 10 primers or pairs of primers) of target-specific primers, between 7 and 100,000 primers or pairs of primers, between 7 and 1000 primers or pairs of primers, between 1,000 to 100,000 primers or pairs of primers, over 100,000 primers or pairs of primers of target-specific primers, etc., between 10 and 100,000 primers or pairs of primers, between 10 and 1000 primers or pairs of primers, etc. Although seven or more primers or pairs of primers are specified and may be preferable, less than seven pairs may be used (e.g., two or more primers or pairs of primers, three or more primers or pairs of primers, four or more primers or pairs of primers, five or more primers or pairs of primers, or six or more primers or pairs of primers, may be used). The target-specific primers may also comprise any appropriate plurality of primers plus any appropriate plurality of pairs of primers, such as 7 primers plus 7 pairs of primers.

The types of primers that may be used may include unmodified oligonucleotides, modified oligonucleotides, peptide nucleic acid (PNA); modified primers may contain one or more than one 5-methyl deoxycytidine and/or 2,6-diaminopurine, dideoxyinosine, dideoxyuridine, and biotin labeled oligonucleotides. One and/or both primers can contain barcodes or other sequences that allow for identification; one and/or both primers can contain adapter sequences.

Equally important to successful amplifying short cDNA fragments by a multiplex primer extension reaction is the efficient removal of non-specific amplification products. Any of the methods described herein may be configured to simultaneously or concurrently degrade non-specific amplification products. In any of the methods described herein, the method may also include removing the degraded non-specific amplification products, leaving the substantial proportion of said plurality of target-specific amplification products. For example, any of the methods described herein may be used with any of the methods or apparatuses described U.S. Pat. No. 9,464,318. Any of the methods described herein may include analyzing the target-specific amplification products. Analyzing may include any appropriate method or technique, including but not limited to sequencing, such as NGS sequencing. Amplification may include any appropriate polynucleotide amplification technique, including in particular a multiplex polymerase chain reaction (PCR).

### BRIEF DESCRIPTION OF THE DRAWINGS

The novel features of the methods and systems described herein are set forth with particularity in the claims that follow. A better understanding of the features and advantages of these methods and systems will be obtained by reference to the following detailed description that sets forth illustrative embodiments, in which the principles of the invention are utilized, and the accompanying drawings of which:

FIG. 1 shows an example of a schematic diagram of a procedure to amplify RNA fragments by using one universal primer at 5′ end and one target specific primer at 3′ end. A universal adapter is added on the 3′ end of the newly synthesized cDNA by template switching while the RNA is converted into cDNA from random hexamers in reverse transcription. The resulting cDNA serves as template for target amplification in multiplex PCR with a universal primer and a panel of target specific primers. Any target specific primer can amplify targets from cDNA of various lengths, as long as this target specific primer finds its complementary site on the cDNA fragments. Target amplification with one target specific primer has higher efficiency than the technologies involves a pair of primers, such as PCR.

FIG. 2 is a model of the efficiency of amplifying short DNA fragments by single-primer and PCR method. The lengths of the DNA fragments are normally distributed in a range of ±33% of the peak. The efficiency of is calculated as eff.=1−(length of amplicon)/(length of DNA peak). The length of amplicon is the summation of the length of primer(s) and the minimal length of the insert. The average length of the primer is 25 nucleotides. The minimal length of the insert for both single-primer method and PCR is 20 bp. To amplify a mixture of DNA fragments with 160 bp peak length, the efficiency of single-primer method is 72%, the efficiency of PCR method is 56%.

FIG. 3 shows an example of a workflow to amplify RNA fragments starting from reverse transcription and template switching. After reverse transcription, the remaining primers and non-specific products are removed in a digestion step. Then a panel of target specific primers and a universal primer are used to amplify targets from the cDNA in a multiplex PCR reaction. The remaining primers and non-specific products are removed again in an additional digestion step. The enriched targets can be further amplified in a PCR reaction to add sample index and sequencing adapters.

FIG. 4 shows the confirmation that intact or long RNA molecules are broken into small fragments. The peak of the curve represents RNA fragments of 100-120 nucleotides in length that were detected at 25 seconds after the migration of RNA fragments was started in a gel.

FIG. 5 shows a library generated from 50 ng of RNA fragments. The cDNA fragments were amplified with a universal primer and a panel of 61 target specific primers, followed by a PCR to add a sample index and sequencing adapters.

FIG. 6 illustrates the 11 fusion mutations detected by sequencing of the library made from 50 ng of Seraseq® Fusion RNA Mix V4 by using the method described in Example 1. The resulting library was sequenced in an Illumina Miseq. The y-axis shows the reads of each mutation detected by NGS, while the genes involved in each fusion mutation are indicated on x-axis.

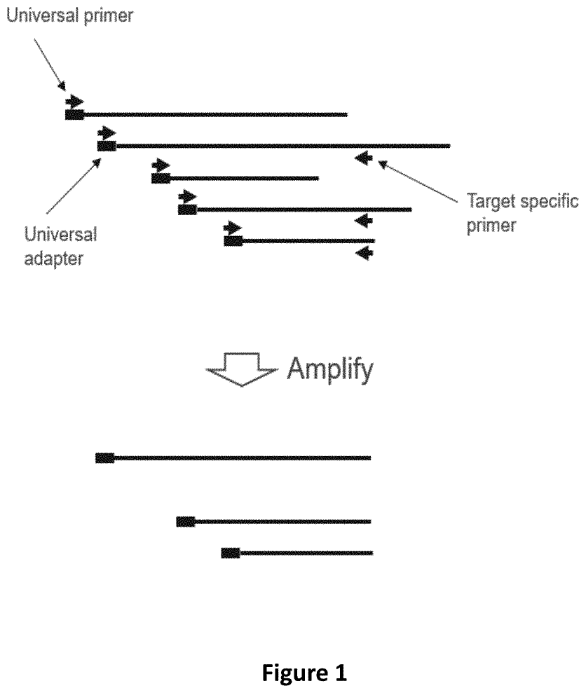

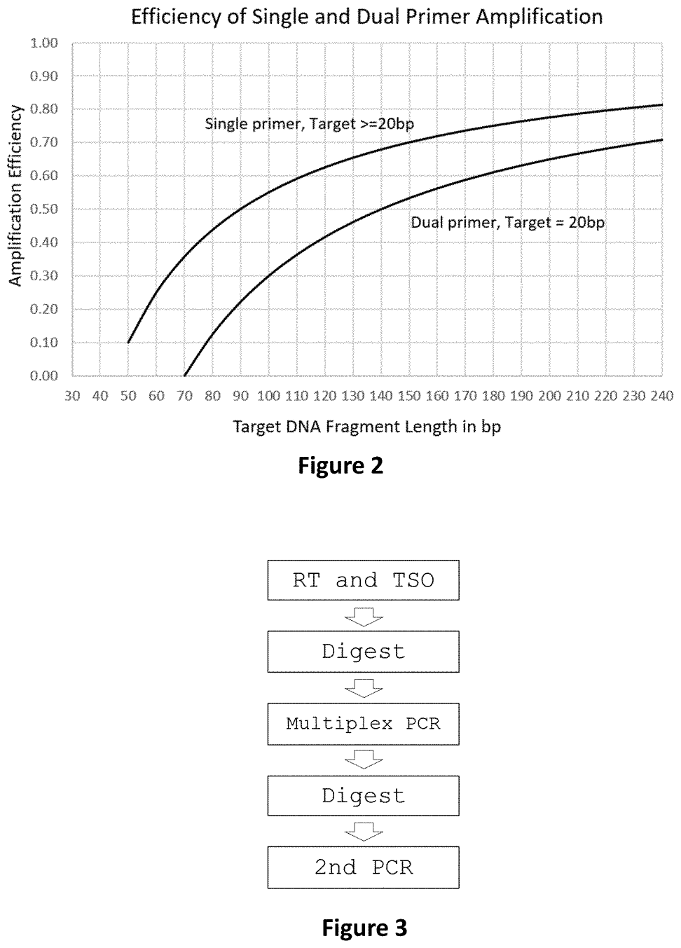

### DETAILED DESCRIPTION

In general, described herein are methods, compositions, systems that may be used to amplify or improve amplification of short target-specific amplification products with reduced number of random errors when amplifying multiple different nucleotide regions. Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Green and Sambrook, Molecular Cloning: A Laboratory Manual (4th Edition, Cold Spring Harbor Laboratory Press, 2012); Rio et al, RNA: A Laboratory Manual (1st Edition, Cold Spring Harbor Laboratory Press, 2010), Berger and Kimmel, Methods in Enzymology, Vol. 152, Guide to Molecular Cloning Techniques (Academic Press, 1987); Alberts et al, Molecular Biology of the Cell (6th Edition, W. W. Norton & Company, 2014), Kornberg and Baker, DNA Replication (2nd Edition, W.H. Freeman, 1992); Nelson and Cox, Lehninger Principles of Biochemistry (7th Edition, W. H. Freeman, 2017); Strachan and Read, Human Molecular Genetics (4th Edition Garland Science, 2010).

“Unique Molecular Identifiers” (UMI) refers to a unique of nucleotide sequence or combination thereof used to label other DNA or RNA molecules. They are usually designed as a string of totally random nucleotides (such as NNNNNNN), partially degenerate nucleotides (such as NNNRNYN), or defined nucleotides (when template molecules are limited). They have given other names, including “molecular barcode”, “molecular index”, “unique identifiers” (UID), “single molecular identifiers” (SMI), “primer ID”, “duplex barcodes”, etc. Molecular barcodes can be as long as 3 to 50 nucleotides, or even longer. They are usually synthesized as a part of the primer or adapter, for example, as a stretch of degenerated nucleotides on either 3′ or 5′ end of adapter. That is, the adapter part has designated nucleotide sequence, the molecular barcode part has random sequences. Molecular barcode can be single stranded, for example, as a part in primer; or double stranded, as it is in adapter. Molecular barcodes are usually added onto the targeted molecules by ligation or through primers during PCR or reverse transcription. Molecular barcodes are used in various applications including, but not limited to, RNA sequencing, studies of single cells, and detection of low frequency mutations. The main purposes of using molecular barcodes are deducing a consensus sequence from the sequences of a group of amplified descendant molecules, thereby to detect the quantity of the original target through removing amplification bias, and finding the true nucleotide sequence of the target through removing random errors and even the false targets. Consensus sequence can be deduced from the amplified sequences of either stand of the target DNA molecule, or collectively from both strands. “Collectively” means the amplified sequences from both of the sense and the antisense strand of the target DNA cannot be differentiated and have to be analyzed together; or the sequences from both strands can be differentiated but be treated as undifferentiated and analyzed together. Complementary double stranded molecular barcodes are used to label both strands of the target molecules, allowing deducing a consensus nucleotide sequence from both strand of the target DNA molecules.

“UMI cluster” means a group of molecular barcodes, on their corresponding target molecules, that have identical or closely related nucleotide sequence. The identical or closely related nucleotide sequence of molecular barcodes of a barcode family is also called as “unique molecular barcode”. “Closely related” means any of the molecular barcodes within one specific family may have one, or two, or three, or any number of different nucleotides, or one, or two, or three, or any number of more or less nucleotides.

“Single strand consensus” means using the sequences from either the sense strand or the antisense strand, or from both of the sense and antisense strand non-discriminatorily of a target DNA molecule to deduce a consensus nucleotide sequence, or the consensus nucleotide sequence deduced from the sequences of either the sense strand or the antisense strand, or from both of the sense and antisense strand non-discriminatorily of the target DNA molecule.

“Double strand consensus” means using the sequences from both of the sense strand and the antisense strand of a target DNA molecule to deduce a consensus nucleotide sequence, or using the sequences from both of a group of the sense strands and a group of the antisense strands of the target DNA molecules to deduce a consensus nucleotide sequence; or the consensus nucleotide sequence deduced from the sequences of the sense strand and the antisense strand of the target DNA molecule, or the consensus nucleotide sequence deduced from the sequences of a group of the sense strands and a group of the antisense strands of the target DNA molecules. Double strand consensus involves, but not limited to, the finding of complementary double stranded molecular barcodes that are used to label both strands of the target molecules, or finding the molecular barcode patterns, as described in the methods and systems described herein, that allows differentiating the sense strands and the antisense strands of the target DNA molecules.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “TAQMAN™” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the methods and systems described herein are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like. The one or more reagents configured for primer extension reaction and exonuclease cleavage described herein may be configured to include components that permit the primer extension and/or exonuclease cleavage to proceed. For example, one or more reagents configured for primer extension reaction and exonuclease cleavage may include buffering agents to maintain pH at a selected level during a reaction, salts, co-factors, etc.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base pairing with a nucleotide in the other strand. A stable duplex can include Watson-Crick base pairing and/or non-Watson-Crick base pairing between the strands of the duplex (where base pairing means the forming hydrogen bonds). In certain embodiments, a non-Watson-Crick base pair includes a nucleoside analog, such as deoxyinosine, 2,6-diaminopurine, PNAs, LNA's and the like. In certain embodiments, a non-Watson-Crick base pair includes a “wobble base”, such as deoxyinosine, 8-oxo-dA, 8-oxo-dG and the like, where by “wobble base” is meant a nucleic acid base that can base pair with a first nucleotide base in a complementary nucleic acid strand but that, when employed as a template strand for nucleic acid synthesis, leads to the incorporation of a second, different nucleotide base into the synthesizing strand (wobble bases are described in further detail below). A “mismatch” in a duplex between two oligonucleotides or polynucleotides means that a pair of nucleotides in the duplex fails to undergo Watson-Crick bonding. “Unmatched base pairs” in a duplex between two oligonucleotides or polynucleotides means that these pairs of nucleotides in the duplex fails to undergo Watson-Crick bonding. A “heteroduplex” region in a duplex between two oligonucleotides or polynucleotides means that the nucleotides on the two strands of this region are unmatched base pairs with each other.

“Kit” refers to any delivery system for delivering materials or reagents for carrying out a method of the methods and systems described herein. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Next-generation sequencing” (NGS) as used herein refers to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (Ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; and Zhang et al., “The impact of next-generation sequencing on genomics”, J Genet Genomics, 2011, 38(3):95-109.

“Nucleotide” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structural Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (“LNAs”), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few nanoliters, e.g. 2 nl, to a few hundred μl, e.g. 200 μl. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“TAQMAN™”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified.

“Primer” or “target specific primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for maximum efficiency in amplification, but may alternatively be double-stranded. If double-stranded, the primer is usually first treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecule in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, biotin-avidin or biotin-streptavidin interactions, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

“Sample” means a quantity of material from a biological, environmental, medical, or patient source in which detection, measurement, or labeling of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The terms “upstream” and “downstream” in describing nucleic acid molecule orientation and/or polymerization are used herein as understood by one of skill in the art. As such, “downstream” generally means proceeding in the 5′ to 3′ direction, i.e., the direction in which a nucleotide polymerase normally extends a sequence, and “upstream” generally means the converse. For example, a first primer that hybridizes “upstream” of a second primer on the same target nucleic acid molecule is located on the 5′ side of the second primer (and thus nucleic acid polymerization from the first primer proceeds towards the second primer).

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The methods provided herein can be used for amplifying a plurality of short RNA fragments. These methods may involve a plurality of DNA primers or oligonucleotides. The methods disclosed herein provide for optimized protocols such that short RNA fragments are amplified. Overall, the methods can relate to improved methods of nucleic acid library preparation.

In one aspect, the methods provide for converting short RNA fragments into cDNA and adding a universal primer at 3′ end of the newly synthesized cDNA by template switching. The methods also provide for amplifying a plurality of targets from the resulting cDNA through the use of a universal primer and a plurality of target specific primers.

The method may involve providing a biological sample comprising at least one target nucleic acid. The nucleic acids can be total RNA, mRNA or FFPE RNA. The RNA can be derived from a eukaryotic cell, an archaea cell, a bacterial cell, a mycobacterial cell, a bacteriophage, a DNA virus, or an RNA virus. In some cases, the RNA can be derived from a mammal. In some cases, the RNA can be derived from a human. The RNA can be intact, or partially degraded, or damaged (e.g., from FFPE samples). The length of the shortest RNA fragment can be 24 nucleotides. The length of the RNA fragments in the plurality can be 30-200, 40-200, 50-200, 60-200, 70-200, 80-200, 90-200, 100-1000, etc. nucleotides in length. Or the length of the RNA fragments in the plurality can be any numbers of combinations as long as the shortest one is equal or longer than the shortest primer, and the longest one is equal or shorter than the length allowed by the cDNA synthesis.

In one aspect, the RNA fragments are converted into cDNA by reverse transcription with random hexamers. Or the RNA fragments are converted into cDNA by reverse transcription with an oligo(dT) primer and random hexamers. Or a poly(A) is added onto the 3′ end of the RNA fragments by poly(A) polymerase before reverse transcription with an oligo(dT) primer. Before poly(A) tailing, the RNA fragments may be treated with T4 polynucleotide kinase to remove 3′-phosphates or 3′-cyclophosphate. Random hexamer and/or oligo(dT) primer may be used at concentrations from 2 to 10 μM.

In one aspect, template switching is used to add a universal primer at the 3′ end of the newly synthesized cDNA. Template switching may be carried out simultaneously with reverse transcription by the same reverse transcriptase. The template switching primer contains a universal primer at 5′ end, followed by 3 consecutive ribonucleotide G (rGrGrG) at the 3′ end. The universal primer is used for downstream primer extension amplification of targets. There may be a unique molecular identifier (UMI) immediately following the universal primer. The UMI region contains 6-40 random nucleotides. The random nucleotides of UMI may be interspersed by fixed nucleotides. The adapter can comprise unmodified bases and/or phosphodiester bonds, or modified bases and/or phosphodiester bonds, unprotected 5′ ends, or protected 5′ ends (such as by biotin), 5′ phosphorylated, or 5′ unphosphorylated ends. Template switching primer may be used at concentrations from 2 to 10 μM.

In one aspect, any reverse transcriptase that supports template switching can be used. These reverse transcriptases include, but not limited to, Superscript II, Protocsript II, SMARTScribe, Maxima H-, RevertAid, EnzScript, GoScript, RevertUP II, MMLV Point Mutant. 20 to 200 units of reverse transcriptase may be used in 20 μl reactions. The reverse transcription may be carried out by first incubation at a lower temperature for 10 to 20 minutes, followed by incubation at a higher temperature for 60 to 90 minutes. The lower temperature may be 8 to 25° C., the higher temperature may be 42 to 55° C.

In one aspect, the methods as disclosed herein can further involve contacting the reverse transcriptase reaction with a 3′→5′ single-stranded DNA specific exonuclease for cleaving single-stranded DNA regions and the primers. As used herein, the term “contacting” equates with introducing such enzyme to a pre-existing mixture as described herein. The methods of the present disclosure can use a variety of single-stranded DNA specific exonucleases that can recognize and cleave single-stranded DNA regions in 3′→5′ direction. The plural form will be used herein to refer to enzymes that bind to and cleave aberrant DNA structures. The single-stranded DNA regions include, without limitation, branched DNAs, Y-structures, heteroduplex loops, single stranded overhangs, mismatches, and other kinds of non-perfectly-matched DNAs. In some examples, the single-stranded DNA specific nuclease can reduce the amount of single-stranded DNA regions in the amplification reaction without reducing the amount of target-specific amplification products that do not contain single-stranded DNA regions. In other examples, both single-stranded DNA regions and target-specific amplification products can be reduced. In some cases, the reaction can be substantially free of single-stranded DNA regions. Substantially free of single-stranded DNA regions can mean that the amount of single-stranded DNA regions in the amplification reaction have been reduced by greater than 50%, 60%, 70%, 80%, 85%, 90%, 91%, 92%, 93%, 94%, 95%, 96%, 97%, 98%, 99%, up to 100%.

Examples of 3′→5′ single-stranded DNA specific exonucleases that can be utilized to cleave single-stranded DNA regions in the methods provided herein include, without limitation, exonuclease T, exonuclease I. It should be understood that essentially any 3′→5′ single-stranded DNA specific exonuclease or its mutant that can perform the methods of the disclosure as described herein is envisioned.

A plurality of target-specific primers is used in a multiplex primer extension reaction. The plurality of target-specific primers selectively enriches a plurality of target nucleic acids. The plurality of target-specific primers can be in primer pairs, or not in pairs, or in a combination of singular primers and primer pairs. The number of the plurality of target-specific primers can be from 7 to over 100,000 primers. In one case, the plurality of target-specific primers comprises at least 7 target-specific primers. In another case, the plurality of target-specific primers comprises from about 7 to about 100 primers. In another case, the plurality of target-specific primers comprises from about 100 to about 1,000 primers. In yet another case, the plurality of target-specific primers comprises from about 1,000 to about 100,000 primers. In a further case, the plurality of target-specific primers comprises over 100,000 primers.

Multiplex PCR reactions as envisioned in this disclosure can be performed by thermostable DNA polymerases commonly used in PCR reactions. Thermostable DNA polymerases can be wild-type, can have 3′→5′, 5′→3′, or both 3′→5′ and 5′→3′ exonuclease activity, or can be a mixture of thermostable polymerases for higher fidelity, or can synthesize long amplicons, or have faster synthesizing rate. An example of a suitable thermostable DNA polymerase can be Taq DNA polymerase. The thermal profile (temperature and time) for the PCR can be optimized, the primer concentration can also be optimized to achieve the best performance. Finally, any additives that can promote optimal amplification of amplicons can be used. These additives include, without limitation, dimethyl sulfoxide, betaine, formamide, Triton X-100, Tween 20, Nonidet P-40, 4-methylmorpholine N-oxide, tetramethylammonium chloride, 7-deaza-2′-deoxyguanosine, L-proline, bovine serum albumin, trehalose, and T4 gene 32 protein.

The methods as disclosed herein can further involve contacting the Multiplex PCR reaction with a 3′→5′ single-stranded DNA specific exonuclease for cleaving single-stranded DNA regions and the primers. As used herein, the term “contacting” equates with introducing such enzyme to a pre-existing mixture as described herein. The methods of the present disclosure can use a variety of single-stranded DNA specific exonucleases that can recognize and cleave single-stranded DNA regions in 3′→5′ direction. The plural form will be used herein to refer to enzymes that bind to and cleave aberrant DNA structures. The single-stranded DNA regions include, without limitation, branched DNAs, Y-structures, heteroduplex loops, single stranded overhangs, mismatches, and other kinds of non-perfectly-matched DNAs. In some examples, the single-stranded DNA specific nuclease can reduce the amount of single-stranded DNA regions in the amplification reaction without reducing the amount of target-specific amplification products that do not contain single-stranded DNA regions. In other examples, both single-stranded DNA regions and target-specific amplification products can be reduced. In some cases, the reaction can be substantially free of single-stranded DNA regions. Substantially free of single-stranded DNA regions can mean that the amount of single-stranded DNA regions in the amplification reaction have been reduced by greater than 50%, 60%, 70%, 80%, 85%, 90%, 91%, 92%, 93%, 94%, 95%, 96%, 97%, 98%, 99%, up to 100%.

Examples of 3′→5′ single-stranded DNA specific exonucleases that can be utilized to cleave single-stranded DNA regions in the methods provided herein include, without limitation, exonuclease T, exonuclease I. It should be understood that essentially any 3′→5′ single-stranded DNA specific exonuclease or its mutant that can perform the methods of the disclosure as described herein is envisioned.

In some cases, the methods as disclosed herein involves the purification of DNA synthesized from primer extension reactions. An example method of RNA purification involves paramagnetic beads, DNA purification column, or precipitation by adding one tenth volume of sodium acetate and two-fold volume of pure ethanol. Another example method of RNA purification involves absorption of DNA onto DNA purification columns and elution afterwards.

In some cases, the amplification products described herein can be used to prepare libraries for next-generation sequencing. The common sequences in the primer pairs are identical to part of adapters useful for next-generation sequencing applications. The adapters can be sequencing adapters useful on a next-generation sequencing platform (e.g., Illumina TruSeq adapters). For example, the methods described herein are useful for next-generation sequencing by the methods commercialized by Illumina, as described in U.S. Pat. No. 5,750,341 (Macevicz); U.S. Pat. No. 6,306,597 (Macevicz); and U.S. Pat. No. 5,969,119 (Macevicz).

Particular reference will now be made to specific aspects and figures of the disclosure. Such aspects are provided by way of example only. Numerous variations, changes, and substitutions will now occur to those skilled in the art without departing from the disclosure.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

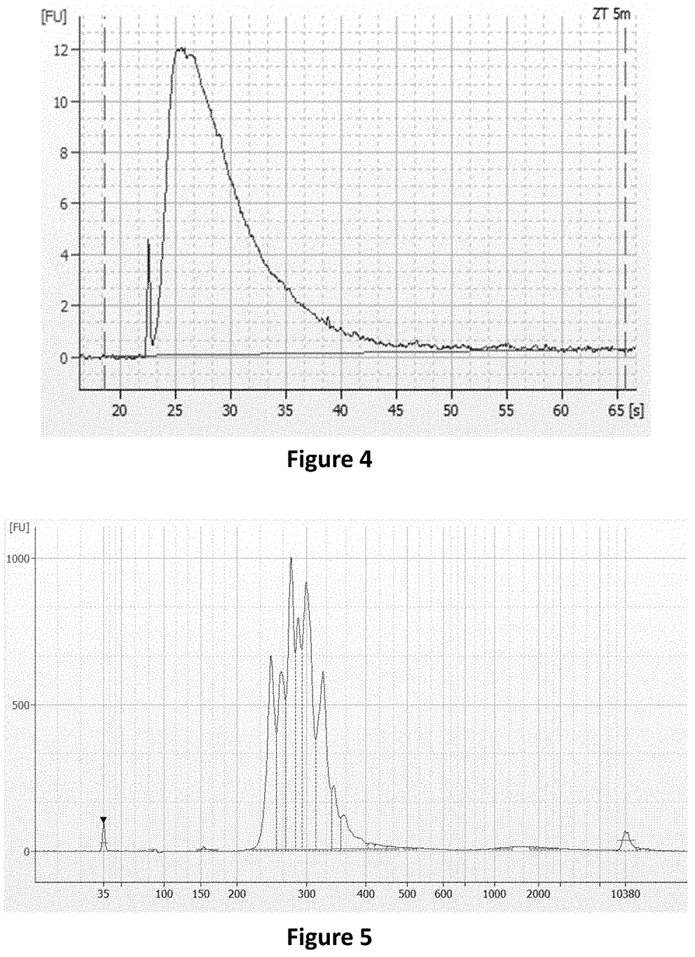

An example of the methods and systems described herein is to amplify 61 targets from short RNA fragments. Short RNA fragments were made by breaking reference RNA (Quantitative PCR Human Reference Total RNA, Agilent, catalog number 750500) into short fragments with NEBNext® Magnesium RNA Fragmentation Module (New England Biolab, catalog number E6150S) according to the suggested method. The lengths of these RNA fragments were confirmed by using 2100 BioAnalyzer instrument (Agilent Technologies, catalog number G2938B) (FIG. 4).

50 ng of RNA fragments were denatured at 65° C. for 5 minutes in the presence of 3 μM of random hexamer and 2 mM of dNTP in 14 μl, followed by immediate incubation on ice for 3 minutes. Then 4 μM of a template switching primer, reverse transcription buffer (50 mM Tris-HCl, pH 8.3 at 25° C., 75 mM KCl, 3 mM MgCl₂, 10 mM DTT) and 200 unites of SMARTScribe™ Reverse Transcriptase (TaKaRa, catalog number 639538) were added into the reaction. The total volume of the reaction was 20 μl. The reverse transcription and template switching reaction was carried out for 10 minutes at 8° C. followed by 80 minutes at 42° C. The sequence of the template switching primer is 5′ Biotin-TTC AGA CGT GTG CTC TTC CGA TCT rGrGrG 3′ (made by Integrated DNA Technologies).

Immediately following the reverse transcription and template switching, 2 μl of the CleanPlex® Digestion Reagent (CleanPlex® Multiplex PCR Kit, Paragon Genomics) was added into the reaction and incubated for 20 minutes at 37° C. The reaction was then stopped with the Stop Buffer in the CleanPlex® Multiplex PCR Kit. The resulting cDNA was purified by using 2.2-fold volume of magnetic beads (CleanMag® Magnetic Beads, Paragon Genomics) by following the user guide. The cDNA was eluted in 10 μl of dH2O.

A panel of 61 target-specific primers and a universal primer were used in a multiplex PCR reaction. The sequences of these 61 primers are list in Table 1, below. Table 1 lists the nucleotide sequences of the 61 target specific primers used as described herein. The sequence of the universal primer is shown as SEQ ID No. 62 (TTC AGA CGT GTG CTC TTC CGA TCT). 3 μM of the universal primer and 5 nM each of the target-specific primers were added into the eluted cDNA, together with Multiplex PCR Master Mix from CleanPlex® Multiplex PCR Kit. The final volume of the multiplex PCR reaction was 20 μl. The multiplex PCR was carried out for 10 cycles with the PCR method suggested in the user guide.

TABLE 1
List of primers of the lung cancer fusion panel

| Sequence ID | Sequence Listing - Primers of |
|---|---|
| number | lung cancer fusion panel |
| SEQ ID NO. 1 | AAGAAGGTGTGTCTTTAATTGAAGCA |
| SEQ ID NO. 2 | AAATGCCCATGAGAGGAAATGG |
| SEQ ID NO. 3 | CCACCCTCTAGGGTTGTCA |
| SEQ ID NO. 4 | GGGAGCTAGAAGTGACGTCTAG |
| SEQ ID NO. 5 | TCACTGATGGAGGAGGTCTTG |
| SEQ ID NO. 6 | CTTGCTCAGCTTGTACTCAGG |
| SEQ ID NO. 7 | GATCTCCATATCCTCCCCTGAG |
| SEQ ID NO. 8 | GGCCCTTGAAGCACTACAC |
| SEQ ID NO. 9 | TAGTCGGTCATGATGGTCGAG |
| SEQ ID NO. 10 | GCTCTGAACCTTTCCATCATACTTAGA |
| SEQ ID NO. 11 | AGTCGGTCATGATGGTCGA |
| SEQ ID NO. 12 | ATCATGATGCCGGAGAAAGC |
| SEQ ID NO. 13 | GCTCAGCTTGTACTCAGGG |
| SEQ ID NO. 14 | CTTGCCAGCAAAGCAGTAGT |
| SEQ ID NO. 15 | TGGTGCTTCCGGCGGTA |
| SEQ ID NO. 16 | CCCTTGAAGCACTACACAGG |
| SEQ ID NO. 17 | GAGCTTGCTCAGCTTGTACTC |
| SEQ ID NO. 18 | GCCAGCAAAGCAGTAGTTGG |
| SEQ ID NO. 19 | GGTGCTTCCGGCGGTAC |
| SEQ ID NO. 20 | GGAGCCAAAGTCAGTCATCAG |
| SEQ ID NO. 21 | TGTAAATTGCCGAGCACGTA |
| SEQ ID NO. 22 | CTGATCAGCCAGGAGGATACA |
| SEQ ID NO. 23 | GCTCCTCCAGGTCTGTGATTA |
| SEQ ID NO. 24 | TGCGTCCTCAAAGGAGACAT |
| SEQ ID NO. 25 | TGCAAAATTCCCTGACGTTGTT |
| SEQ ID NO. 26 | GTCCCAGTGGTGGATGTAGAT |
| SEQ ID NO. 27 | TTGTCCCAGTGGTGGATGTA |
| SEQ ID NO. 28 | GAGCAGCGTAGAAAGGAAGAG |
| SEQ ID NO. 29 | GCTGAGGTTGTAGCACTCG |
| SEQ ID NO. 30 | GGTGATGCCGTGGTTGATG |
| SEQ ID NO. 31 | GCAGAGGATTAGGCTCAGC |
| SEQ ID NO. 32 | GCACCGAGACGATGAAGGA |
| SEQ ID NO. 33 | AATTTGATGACATGTGGGTGGT |
| SEQ ID NO. 34 | ACTTTTCCAAATTCGCCTTCTCC |
| SEQ ID NO. 35 | CACTTTTCCAAATTCGCCTTCTC |
| SEQ ID NO. 36 | CCACTTTTCCAAATTCGCCTTCT |
| SEQ ID NO. 37 | CTTTTCCAAATTCGCCTTCTCCTA |
| SEQ ID NO. 38 | CTTTTCCAAATTCGCCTTCTCCT |
| SEQ ID NO. 39 | GGGAACCCACAGTCAAGGT |
| SEQ ID NO. 40 | TTATCTTCAGCTTTCTCCCACTGTA |
| SEQ ID NO. 41 | CAGCCAACTCTTTGTCTTCGTTTAT |
| SEQ ID NO. 42 | TCAGCTTTCTCCCACTGTATTGA |
| SEQ ID NO. 43 | ATTCCCTTTGAGGTTTTTACTGCAT |
| SEQ ID NO. 44 | GGTCAGTGGGATTGTAACAACC |
| SEQ ID NO. 45 | CAGCTTTCTCCCACTGTATTGAA |
| SEQ ID NO. 46 | CAGTGGGATTGTAACAACCAGAAA |
| SEQ ID NO. 47 | CCAACTCTTTGTCTTCGTTTATAAGC |
| SEQ ID NO. 48 | GTCAGTGGGATTGTAACAACCAG |
| SEQ ID NO. 49 | CAAGAGACGCAGAGTCAGT |
| SEQ ID NO. 50 | CCAAGTTTCTCACTTGGAATACTACAA |
| SEQ ID NO. 51 | TCAGCCAACTCTTTGTCTTCG |
| SEQ ID NO. 52 | TCAGTGGGATTGTAACAACCAGA |
| SEQ ID NO. 53 | CAGTGGGATTGTAACAACCAGAA |
| SEQ ID NO. 54 | CTTCATACACTTCTCCAAAGGCT |
| SEQ ID NO. 55 | CTTCAGCTTTCTCCCACTGTATTG |
| SEQ ID NO. 56 | GACCATCTGGCGACGGT |
| SEQ ID NO. 57 | GTCTACCAGGACTGTCCCTC |
| SEQ ID NO. 58 | CCTCTTCGAACCTGTCCATGAT |
| SEQ ID NO. 59 | GTACTGGAGCAGGTCCACTATA |
| SEQ ID NO. 60 | GTGTTTTCTTCAACCAAAGCAGTTTAT |
| SEQ ID NO. 61 | GCATGAACCGTTCTGAGATGAAT |
| | Universal primer: |
| SEQ ID NO. 62 | TTCAGACGTGTGCTCTTCCGATCT |

After the multiplex PCR, the reaction was stopped with the Stop Buffer. The DNA was purified with 1.3× CleanMag® magnetic beads. 1 μl of CleanPlex® Digestion Reagent was used to remove the primers and non-specific products for 10 minutes at 37° C. The DNA was then purified again with 1.3× CleanMag® magnetic beads by following the user guide. The purified DNA was subjected to one more round of PCR for 22 cycles with primers containing Illumine sequencing adapters. After the PCR, the DNA was purified by using 1-fold volume of magnetic beads (CleanMag® Magnetic Beads) to generate the library.

The size, concentration and purity of this library were assayed in a 2100 BioAnalyzer instrument (Agilent Technologies, catalog number G2938B). 1 μl of each library was assayed with a high sensitivity DNA analysis kit (Agilent Technologies, catalog number 5067-4626), according to the methods provided by the supplier. The results are presented in FIG. 5. Upon sequencing this library by NGS, we found that the on-target rate was 93%, ribosomal RNA rate was less than 1%. We thus demonstrated that a clean library was made by an example method of this invention.

### Example 2

In this example, a library was made by using the same method described in Example 1. However, a mixture of RNA fragments containing known mutations was used in order to validate detection of these mutations by sequencing the made library by NGS. Thus, the reference RNA fragment was replaced with Seraseq® Fusion RNA Mix V4 (SeraCare, Material Number 0710-0496). There are 18 known fusion mutations in Seraseq® Fusion RNA Mix V4, 11 of the fusion mutations can be amplified and detected by the panel of 61 target-specific primers. The 11 known fusion mutation are listed in Table 2.

TABLE 2
fusion mutations existing in Seraseq® Fusion RNA
Mix V4 covered by the
target specific primers from Table 1.
Fusions existing in Seraseq® Fusion RNA Mix V4 and
covered by the panel of primers described herein

NCOA4 | 51582940 | RET | 43612031
KIF5B | 32306070 | RET | 43609927
EML4 | 42522657 | ALK | 29446395
FGFR3 | 1808662 | TACC3 | 1741428
CD74 | 149784242 | ROS1 | 117645579
SLC34A2 | 25665953 | ROS1 |117645579
TPM3 | 154142875 | NTRK1 | 156844362
LMNA | 156100565 | NTRK1 | 156844697
CCDC6 | 61665879 | RET | 43612031
ETV6 | 12022904 | NTRK3 | 88483985
TFG | 100451517 | NTRK1 | 156844362

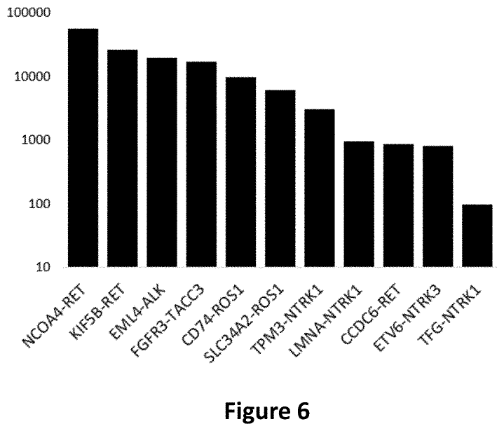

50 ng of Seraseq® Fusion RNA Mix V4 was used to make a library by using the method described in Example 1. The resulting library was sequenced in an Illumina Miseq. Again, we found that the on-target rate was 96%, ribosomal RNA rate was less than 1%. All 11 fusion mutations were detected in FIG. 6, which shows the reads of each mutation detected by NGS.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of amplifying targets from a plurality of RNA fragments by using a multiplex primer extension reaction, the method comprising:
designing a plurality of target specific primers to be used in a multiplex primer extension reaction, wherein the 5′ end of said plurality of target specific primers contain an adapter sequence;
designing a template switching oligo, wherein said template switching oligo comprises, from 5′ to a 3′ end, an adapter sequence, a stretch of random nucleotide sequences to be used as unique molecular identifier, and a three-base RNA sequence of CCC;
converting an RNA into a single-stranded cDNA by using reverse transcriptase, random hexamer and the template switching oligo to form a cDNA synthesis reaction;
removing single-stranded DNA in the cDNA synthesis reaction through enzymatic digestion; and
amplifying a plurality of targets from the cDNA synthesis reaction using the plurality of target specific primers and the adapter sequence in a multiplex primer extension reaction, followed by enzymatic removal of nonspecific amplification products.

2. The method of claim 1, further comprising further amplifying the products in a primer extension reaction.

3. The method of claim 1, wherein the plurality of RNA fragments is total RNA, mRNA, fragmented total RNA, fragmented mRNA, fragmented total RNA purified from Formalin-fixed, Paraffin-embedded (FFPE) tissue samples (FFPE RNA), and RNA fragments purified from plasma.

4. The method of claim 1, wherein the multiplex primer extension reaction comprises multiplex polymerase chain reaction.

5. The method of claim 1, wherein the plurality of target-specific primers includes a target-specific region that is complimentary to the plurality of target RNA.

6. The method of claim 1, wherein the plurality of target specific primers comprises either forward primers or reverse primers.

7. The method of claim 1, wherein the plurality of target specific primers comprises both forward primers and reverse primers.

8. The method of claim 1, wherein each primer of the plurality of target specific primers includes a target specific region that is from 8-50 nucleotides.

9. The method of claim 1, wherein said plurality of target specific primers comprise between 7 target specific primers and 1,000,000 target specific primers.

10. The method of claim 1, wherein each primer of the plurality of target specific primers includes a target specific region comprising unmodified oligonucleotides.

11. The method of claim 1, wherein each primer of the plurality of target specific primers includes a target-specific region comprising modified oligonucleotides with chemical modifications of nucleotides.

12. The method of claim 1, wherein the adapter sequence comprises a region of nucleotide sequence used for further amplification and for high-throughput sequencing.

13. The method of claim 1, wherein the primer and/or template switching oligo contains a unique molecular index comprising 12-40 random nucleotides.

14. The method of claim 1, wherein the reverse transcriptase is one or more of: GoScript™ reverse transcriptase, Maxima H minus reverse transcriptase, ProtoScript® II reverse transcriptase, RevertAid reverse transcriptase, SMARTScribe™ reverse transcriptase, and Superscript® II reverse transcriptase.

15. The method of claim 1, wherein the single-stranded cDNA is synthesized by using 0.2-2 uM of oligo(dT), 1-10 uM of hexamer primer and 10-200 units of reverse transcriptase at 42° C. for 90 minutes.

16. The method of claim 1, wherein the reverse transcription reaction is treated by using an exonuclease, multiple exonucleases, or a combination of exonucleases and nucleases, selected from the group comprising: S1 nuclease, P1 nuclease, mung bean nuclease, lambda exonuclease, exonuclease I, exonuclease VII, exonuclease T, RecJ, RecJf.

17. The method of claim 1, further comprising purifying the synthesized DNA by using magnetic beads or a DNA purification column.

18. The method of claim 1, wherein the multiplex primer extension reaction is a multiplex polymerase chain reaction.

19. The method of claim 1, wherein the multiplex primer extension reaction is further treated by using an exonuclease, multiple exonucleases, or a combination of exonucleases and nucleases, selected from the group comprising: S1 nuclease, P1 nuclease, mung bean nuclease, lambda exonuclease, exonuclease I, exonuclease VII, exonuclease T, RecJ, RecJf.

20. The method of claim 19, further comprising amplifying the products of the multiplex polymerase chain reaction with a pair of primers that are complimentary to the adapter sequences by polymerase chain reaction.

21. The method of claim 20, further comprising analyzing the amplification products by high-throughput sequencing.
